(19) United States
(12) Patent Application Publication
(10) Pub. No.: US 2020/0146546 A1
(43) Pub. Date: May 14, 2020

(54) Method for Determining a Light Filter Applied to a Spectacle Lens, Associated Display Device and Virtual Reality Helmet

(71) Applicant: Essilor International, Charenton-Le-Pont (FR)

(72) Inventors: Sylvain CHENE, Charenton-le-Pont (FR); Amandine DEBIEUVRE, Charenton-le-Pont (FR); Marie DUBAIL, Charenton-le-Pont (FR); Sarah MARIE, Charenton-le-Pont (FR); Susana MONTECELO, Charenton-le-Pont (FR); Anne-Catherine SCHERLEN, Charenton-le-Pont (FR); Frédéric ARROUY, Charenton-le-Pont (FR)

(21) Appl. No.: 16/609,060

(22) PCT Filed: Apr. 26, 2018
(86) PCT No.: PCT/FR2018/051067

§ 371 Date: Oct. 28, 2019

(30) Foreign Application Priority Data
Apr. 28, 2017 (FR) 1753825

(51) Int. Cl. A61B 3/06; A61B 3/024; A61B 3/08; A61B 3/113; A61B 3/032; A61B 3/00

(52) U.S. Cl. CPC A61B 3/06; A61B 3/066; A61B 3/024; G02C 7/10; A61B 3/113; A61B 3/032; A61B 3/0008; A61B 3/08

(57) ABSTRACT

The invention concerns a method for determining a light filter for a spectacle lens for improving or maintaining the visual comfort of an individual wearing spectacles, comprising the steps: a) collecting data relating to the needs or uses of the individual; b1) selecting a test filter on the basis of a predefined criterion; b2) selecting, as a function of the collected data, a luminous scene comprising an image determined as a function of the needs or uses, and a light source generating a visual discomfort for the individual not equipped with a filter; c) placing of the individual by presenting him with the luminous scene through the test filter; d) evaluating said criterion; e) comparing the result with a predetermined threshold; f) determining the suitable light filter as a function of the result. The invention also concerns a display device and a virtual reality helmet designed for said method.

### TECHNICAL FIELD OF THE INVENTION

The present invention generally relates to the field of ophthalmic optics.

It in particular relates to the field of the prescription of a light filter to an individual who wears spectacles with said filter.

It more particularly relates to a method for determining a light filter suitable for being applied to a spectacle lens in order to improve or maintain the visual comfort and/or the visual performance of this individual.

It also relates to a displaying device and to a virtual reality headset that are suitable for implementing this method.

### BACKGROUND

Generally, an individual who needs to determine which is the best filter with respect to his needs and habits makes his choice on the basis of mainly esthetic (color or tint of the lens, design of the frame, etc.) or financial (price of the lens receiving the filter and/or of the frame intended to receive the lens) criteria.

For a pair of prescription sunglasses for example, one of the rare technical criteria also considered by the individual relates to the “class” of the lens, such as defined by standard NF EN ISO 12312-1. This class may range: from 0 for a “clear” lens that transmits between 80 and 100% of visible light between 380 nanometers (nm) and 780 nm and 8 to 10% of ultraviolet (UV) light in the “UVB” range between 280 nm and 315 nm; to 4 for an extremely dark lens that transmits only between 3 and 8% of visible light and less than 0.3 to 0.8% of UVB light.

However, esthetic and financial criteria or indeed the class of the filter are not really relevant to enabling the individual to determine the most suitable light filter to improve or maintain his visual comfort and/or his visual performance.

Specifically, each individual, whether he is young or old, and depending on his lifestyle (indoors or outdoors, work on a display screen or of meticulous nature, etc.), on the luminous conditions with which he is most frequently confronted (very bright environment, night-time environment, etc.), on his particular needs (need to be protected from glare due to point sources, need to protect his visual acuity), etc., has a sensitivity that is specific thereto.

Moreover, the prescription of a light filter for an individual, whether it be a question inter alia of solar filters (for example tinted sunglasses) or indeed of filters taking the form of a specific spectacle-lens coating (for example an antireflection coating or indeed an anti-UV treatment), is a task that is technically difficult for eye care professionals (opticians, optometrists, etc.).

Specifically, the prescription of a light filter for an individual generally requires many measurements relating to the specific sensitivity of the wearer for whom said filter is intended.

From patent document FR 1650383 in the name of the applicant, a method for determining a filter for an ophthalmic lens intended to be placed in front of the eye of a wearer, said filter being able to improve or to maintain the visual comfort and/or visual performance of said wearer, is in particular known.

The determining method of document FR 1650383 comprises a step of measuring a quantity representative of a sensitivity of the eye of the wearer to a characteristic light flux, and a step of determining at least one optical characteristic of said filter depending on the measured representative quantity.

By “characteristic light flux”, what document FR 1650383 means is:
either a “real” light flux to which the wearer is subjected during a given task: the characteristic light flux is then characteristic of the ambient luminous environment in which the wearer will be when performing the visual task;
or to an “artificial” light flux in the sense that it at least partially reproduces the light flux to which the wearer will be subjected: the characteristic light flux is then representative of at least one light source that causes the wearer visual discomfort or to lose visual performance.

Although the method of document FR 1650383 allows the one or more optical characteristics of one or more filters entirely suitable for the wearer to be precisely and objectively determined, it is however tedious to implement, in particular because it requires a characteristic light flux able to test the sensitivity of the eye of the wearer to light to be generated.

Specifically, as document FR 1650383 teaches, the complexity of the determination of the filter resides in the fact that this sensitivity to light of the eye of the wearer is dependent both on the physical or optical characteristics of the characteristic light flux, on the physiology of the visual system of the wearer, and on the functional impact of a discomforting light flux on the visual performance or the visual comfort of the wearer during a given visual task.

In addition, the interpretation of the sensitivity measurements, in particular the physiological data, with a view to determining which is the right light filter requires a lot of experience on the part of the optician.

### SUMMARY OF THE INVENTION

In order to remedy the aforementioned drawback of the prior art, the present invention proposes a method allowing which is the optimal light filter for an individual to be determined without requiring specific complex measurements relating to the sensitivity of said individual to light or to a type of light to be taken.

Another objective of the invention is to assist an eye care professional with the recommendation of a suitable light filter and the demonstration of its benefit and the rapid testing thereof.

More particularly, according to the invention a method for determining a light filter suitable for being applied to a spectacle lens in order to improve or maintain the visual comfort and/or the visual performance of an individual who wears said spectacles is provided, said method comprising:

a) a step of collecting data relating to the needs and/or the habits of said individual;

b1) a selecting step in which at least one trial filter is selected based on at least one predefined evaluation criterion;

b2) a selecting step in which, depending on said data collected in step a), at least one luminous scene is selected, said scene comprising:
an image or a film determined depending on the visual needs and/or visual habits of the individual; and
at least one light source that generates a discomfort and/or a loss of visual performance for the individual not equipped with filter;

c) a step of exposing said individual to a situation, in which step said luminous scene selected in step b2) is visually presented to said individual through said trial filter selected in step b1);

d) a step of evaluating said predefined criterion of step b1) during the situation exposure of step c); and

e) a step of comparing the result of the evaluation of step d) with a predetermined visual-comfort and/or visual-performance threshold,

f) a step of determining said suitable light filter depending on the result of the comparison of step e).

Thus, by virtue of the invention, it is possible to precisely determine under realistic conditions a light filter for the individual.

The invention has the advantage of exposing the future wearer of the filter to a predetermined luminous environment with a given scenario chosen depending on his needs or habits, and of proposing thereto a realistic rendering of the trial filter, i.e. of really immersing him (case of a real trial filter) or of virtually immersing him (case of a simulated virtual trial filter) in visual conditions that are identical or almost identical to those that he would actually experience in real life with this trial filter.

The invention defines a set of steps of exposure to a situation, which makes it possible to evaluate, according to one or more predetermined criteria, the rendering and/or the benefit achieved with the selected trial filter, to assist with the prescription of one or more optimal light filters, and to differentiate between said filters.

The evaluation on the basis of one or more criteria defined beforehand depending on needs or habits allows the right light filter to be suitably prescribed to the individual.

By virtue thereof, a new service may be offered by eye care professionals who can easily propose and personalize light filters depending on the visual sensitivity of their customer.

By data relating to the “habits” of the individual, what is meant is any data relating to the uses that the individual will possibly make of his light filter. These data are for example related to the conditions of use of the light filter:
use indoors or outdoors;
use during the day or at night;
intermittent or prolonged use, frequency and duration of use;
use for driving a vehicle or a machine;
use in a specific place: sea or mountain (light that is more or less polarized), a generally very sunny place or indeed an often cloudy place, a place where the ambient temperature is high/low, etc.;
use in combination with another particular visual device: safety glasses, magnifying glasses, etc.;
use for a specific task or activity: sport, television, reading/writing, driving a motor vehicle, work on a display screen, manual work, etc.

By data relating to the “needs” of the individual, what is meant is any data relating to the visual discomforts or deficiencies of the individual, and optionally to the symptoms that are associated therewith, and that the sought-after light filter is supposed to palliate, such as those mentioned nonlimitingly below:
visual discomfort;
sensitivity to glare for a certain type of light source: relatively bright source, point or extended source, direct or indirect source, polarized or unpolarized source, continuous or transitory source, diffuse or angularly selective source;
sensitivity to light under particular conditions: day or night, with or without visual correction, etc.;
discomfort due to a light source emitting in a given wavelength range: ultraviolet, visible, blue light between 400 nm and 460 nm, etc.;
loss of perception: of contrasts, of movements, of depth, etc.;
problem with mono- or binocular vision, such as a pathology such as age-related macular degeneration (ARMD) or glaucoma;
decrease in the central and/or peripheral attentional visual field;
difficulty with the recognition of shapes or objects;
degradation of visual acuity, ametropia;
narrowing of the visual field;
decrease in reading performance;
visual fatigue;
reaction time of the visual system, latency or recovery time of the pupil, pupillary response;
poor perception of color; or
migraines, epileptic fits.

According to certain embodiments of the method of the invention, the image (or the film) and the light source may be on one and the same medium, i.e. be generated by the same device, for example a high-dynamic-range (HDR) screen.

According to other embodiments, the image and the source may be on different media, i.e. be generated by two different luminous devices, for example by a conventional liquid-crystal display (LCD) and by a backlight unit, respectively.

Preferably, in step a), said individual is asked to respond to a questionnaire containing questions relating to the needs and/or habits of the individual (see above).

The responses to the questions of this questionnaire are then the data collected in step a).

Advantageously, it is also possible to collect, in step a), measurements relating to the luminous environment of the individual by means of at least one light sensor.

Nonlimitingly, what is meant by measurements relating to the luminous environment of the individual is any physical, in particular optical, measurement of the visual luminance perceived by the individual, of the retinal illuminance, of the spectrum of the ambient light, of the angular and/or spatial distribution of the light sources of the environment, etc.

Said light sensor comprises any sensor sensitive to the (luminous) power, to the luminance, to the illuminance, to the spectrum or to the color temperature of these light sources.

Next, in step b1), said trial filter is selected on the basis of a predefined evaluation criterion, which criterion may be objective or subjective.

Advantageously, said trial filter is selected in step b1) depending on said data collected in step a). This makes it possible to make a relevant preselection of the trial filter.

Preferably, said predetermined criterion is chosen from one of the following filter-evaluation criteria or according to a combination of a plurality of these criteria: visual comfort, sensitivity to glare, perception of colors, perception of contrasts, visual acuity, perception of movements, perception of depth, visual field, reading performance, visual fatigue, reaction time, shape detection and/or recognition, pupillary response, central and/or peripheral attentional visual field.

An example of a combination of two of these criteria is visual acuity measured at low contrast.

The trial filter may also be chosen in step b1) depending on a particular optical characteristic, for example depending on one of the following parameters:
a visual- or light-transmission level for at least one wavelength or a polarization state of the light, for a wavelength band, or for all the visible domain for example;
a spectral response or a reflection and/or transmission spectrum in a given wavelength band, for example in the UV (UVA and/or UVB) or visible domain;
a spatial variation in the above parameters over the area of said lens intended to receive the light filter;
a temporal variation in these parameters over time; or
a variation in the tint or in the absorption level as a function of a UV light flux or of an electrical variable (current, voltage).

Before or after step b1), provision is made for a second selecting step b2) in which at least one luminous scene is generated, said luminous scene comprising:
an image or a film determined depending on the visual needs and/or the visual habits of the individual; and
at least one light source that generates a discomfort and/or a loss of visual performance for the individual.

Preferably, the image or film determined depending on the visual needs and/or visual habits ascertained in step a) corresponds to an image or film that is representative:
of the expected use of the light filter;
of the luminous environments in which the wearer is liable to be when wearing his light filter; and/or
of the discomforts encountered and reported by the individual.

For example, if in step a) of the method a datum is collected that indicates that the wearer will wear his one or more filters when he is working at night, the image or film will represent a night-time situation or a situation in which the visual-luminance spectrum and/or level is low and/or shifted to the blue.

Also by way of example, if the individual reports that he will mainly use his filter when he is driving his motor vehicle, provision will then be made for an image or film representing a driving situation (for example with signs or traffic lights of various colors/shapes/sizes/readability levels, other vehicles coming from the opposite direction with dipped lights turned on, etc.)

Again by way of example, if the wearer complains of discomfort related to an excessively high sensitivity to glare (with for example problems due to a high retinal recovery time or to loss of visual acuity) whatever the situation or his activity, provision will then be made to select a luminous scene in which the image will represent a particular visual test allowing his retinal recovery time or visual acuity to be estimated.

In the same way, the one or more light sources that generate discomfort and/or a loss of visual performance for the individual are representative of the sources of visual discomfort reported in step a).

For example, if in step a) a datum is collected that indicates that the individual is essentially discomforted by direct light sources at night, the additional light source in the luminous scene (comprising an image or a film representative of a night-time situation) will be characteristic of a direct light source (for example the headlights of an automobile, the light of a streetlight, etc.).

At the end of the selecting steps b1) and b2) of the method of the invention, provision is then made, in step c), to view the rendering or the benefit of the one or more trial filters selected according to various predefined evaluation criteria.

In other words, said luminous scene selected in step b2) is visually presented to the individual (i.e. it is placed before or in front of the eyes of the individual) through said trial filter selected in step b1).

Preferably, in step c), said luminous scene selected in step b2) is displayed by a displaying device, and said individual actually wears said trial filter or else wears said trial filter virtually, said luminous scene then being displayed by said displaying device such as it would be seen by said individual if he were actually wearing said trial filter, and said individual observes said displaying device.

In other words, in one particular embodiment, in step c), said luminous scenes selected in step b2) is displayed by a displaying device; and said individual physically wears said trial filter in order to observe said displaying device.

In another preferred embodiment, in step c), said luminous scene viewed through said trial filter selected in step b1) is simulated and displayed by a displaying device such as said luminous scene would be seen by said individual if he were wearing said trial filter virtually; and said individual observes said displaying device. In this case, the effect of the trial filter is viewed indirectly via the display of the simulated and displayed visual scene.

The aim of step d) is to allow the individual who is (actually or virtually) wearing the trial filter to evaluate the benefit of the trial filter according to the criteria selected in step b1). It is a question of qualitatively and quantitatively evaluating the visual comfort of, the visual performance of or various other indicators specific to the wearer in a given situation. The subject may but need not be equipped with any dioptric lenses.

The objective is to define filter-evaluation criteria that are either based on generic tests, or based on the type of environment or the needs identified by the wearer.

The evaluation criteria may be objective, subjective or mixed. For each of the criteria, the result of the evaluation of step d) is compared with a predetermined visual-comfort and/or visual-performance threshold.

Depending on the needs and habits of the wearer, or indeed depending on the predefined evaluation criterion, in step e), the predetermined visual-comfort and/or visual-performance threshold is either an absolute threshold or indeed a relative threshold determined based on a reference filter or based on the trial filter tested beforehand, or indeed based on a situation in which the individual wears no filter.

The reference filter may be a median filter in the sense that it meets the visual needs of half of a representative population of individuals.

The reference filter may even be:
the filter that the wearer usually wears (current filter);
the filter that delivers the best performance with respect to the chosen criterion; or
the last filter made available on the market.

The final step of determining the light filter allows the prescription of the filter to be validated or invalidated.

According to one particular embodiment of the invention, in step f), said trial filter is adopted as being said suitable light filter if the comparison of step e) demonstrates an improvement in said criterion defined beforehand in step b1).

According to another particular embodiment of the invention, if the comparison of step e) demonstrates, in contrast, a degradation in the criterion defined beforehand in step b1), the determining method is restarted, after step f), from step b1) with another trial filter being selected according to the same predefined evaluation criterion or indeed according to another predefined evaluation criterion. In this case, provision may be made, in step b2), to select an identical luminous scene or indeed a different luminous scene, for example if a new predefined evaluation criterion was selected.

Thus, the determining method may be an iterative method that tends toward the light filter that is optimal for the individual depending on his needs and/or habits.

In order to implement the determining method described above, the invention also relates to a displaying device intended to test the visual comfort and/or the visual performance of an individual, comprising a graphical screen that is controlled by a microprocessor in order to display, on said graphical screen, an image or a film capable of being viewed by said tested individual.

According to the invention, said displaying device furthermore comprises an additional illuminating system that is controlled by another microprocessor or by the same microprocessor in order to transmit, to said individual observing said graphical screen, an additional light flux having an energy, a spectral distribution, a temporal variation, a spatial distribution, and/or an angular distribution that is or are predetermined, said additional illuminating system being arranged with respect to said graphical screen so as to let all or some of said image or said film be seen.

The displaying device is particularly suitable for implementing certain embodiments of the aforementioned determining method.

Preferably, said displaying device observed by the individual comprises an augmented reality system, a virtual reality system, an image-projecting system, or indeed a graphical screen combined with an additional illuminating system.

In one particularly advantageous embodiment, said additional illuminating system of the displaying device according to the invention is suitable for transmitting an additional light flux allowing, in combination with said graphical screen, a visual luminance suitable for creating a visual discomfort for the individual, in particular a discomfort due to glare, to be achieved.

Preferably, the visual luminance achieved, by virtue of the addition of the luminous scene displayed by the graphical screen and the additional light flux transmitted by the additional illuminating system, is higher than or equal to 1000 candelas per square meter (cd/m²), and better still higher than or equal to one of the following values: 1500, 2000, 3000 cd/m², and even better still higher than or equal to 4000 cd/m².

In one embodiment, the additional luminous system alone leads to a visual luminance in the range extending from 1000 cd/m² to 20000 cd/m², preferably from 2000 cd/m² to 20000 cd/m², and better still from 3000 cd/m² to 20000 cd/m².

Again preferably, the visual luminance achieved by virtue of the additional illuminating system is that obtained by virtue of an extended and substantially planar light source that has a light-emitting area able to achieve an illuminance higher than or equal to 10000 lux.

Generally, it is difficult to find graphical screens of reasonable price and bulk that display a visual luminance higher than 900 cd/m², the standard visual luminance being about 300 to 500 cd/m².

Thus, by virtue of the displaying device of the invention, it is possible to associate a graphical screen intended to display the image of the visual scene of the method and an additional illuminating system allowing, in combination with the graphical screen or preferably alone, visual luminance levels that are very high, or in any case sufficiently high to generate conditions that cause a standard individual to experience glare, to be achieved.

It is a question of allowing an individual to experience, by virtue of the displaying device, as realistic as possible a simulation of a luminous environment.

Present-day displaying devices do not allow very high light flux to be correctly simulated because of the limited brightness of their graphical screen.

It is therefore not possible to realistically demonstrate to the individual the effects of a filter, nor to precisely determine his sensitivity to a high light level.

The additional illuminating system of the displaying device may include at least one light source that comprises light-emitting diodes (LEDs), optical fibers, or organic light-emitting diodes (OLEDs).

The displaying device may also comprise means for fastening the additional illuminating system to the graphical screen, for example clip-fastening means.

Preferably, the additional illuminating system allows all or some of the information (image, film, etc.) displayed on the graphical screen of the displaying device to be seen.

The following are other nonlimiting and advantageous features of the displaying device according to the invention:
said additional illuminating system comprises light-emitting diodes and an active or passive scattering film placed between the eyes of said individual and said graphical screen, said scattering film being suitable for backscattering the light flux emitted by said light-emitting diodes;
said additional illuminating system comprises a backlight unit placed in front of said graphical screen, and an active or passive transmission-mode scatterer that is placed between said backlight unit and the eyes of said individual;
said illuminating system comprises at least one light-emitting diode and a half-silvered mirror placed between the eyes of said individual and said graphical screen in order to reflect the light flux emitted by said light-emitting diode toward the eyes of the individual;
said displaying device comprises at least two light sensors that are suitable for delivering a signal representative of the illuminance level at the two eyes of said individual, and wherein the average luminance level of said image or of said film and/or of said additional light flux is set depending on this representative signal.

The invention lastly provides a virtual reality headset intended to be worn by an individual, comprising:
a displaying device such as defined above;
means for keeping this displaying device in front of the eyes of said individual; and
means for isolating said individual from ambient light.

Preferably, said microprocessor of the headset is suitable for displaying an image (or a film) formed from a left image (film) for the left eye and a right image (film) for the right eye of said individual, and the headset also comprises optical means for generating three-dimensional imagery, said means being suitable for presenting said left image to the left eye of the individual and said right image to the right eye of the individual, respectively, so that said individual sees a three-dimensional image or film by fusing said left and right images (films).

These optical means for generating three-dimensional imagery may for example comprise two thin lenses the optical axes of which are parallel and separated by a fixed or variable distance, for example equal to the interpupillary distance of the individual.

These optical means for generating three-dimensional imagery may comprise two groups of at least two lenses allowing the optical power of said group to be adjusted, for example in order to tailor it to the refraction of the individual, with or without spectacles.

These optical means for generating three-dimensional imagery could also simply comprise two circular apertures suitable for each of the two eyes of the individual and a substantially planar partition placed between the two apertures, and extending perpendicular to the segment joining two particular points of the eyes of the individual, for example the rotation center of the right eye and the rotation center of the left eye.

### DETAILED DESCRIPTION OF ONE EXAMPLE EMBODIMENT

The description which follows with reference to the appended drawings, which are given by way of nonlimiting examples, will make it easy to understand what the invention consists of and how it may be achieved.

In the appended drawings:

FIG. 1 is a schematic diagram showing the various steps of the determining method according to the invention;

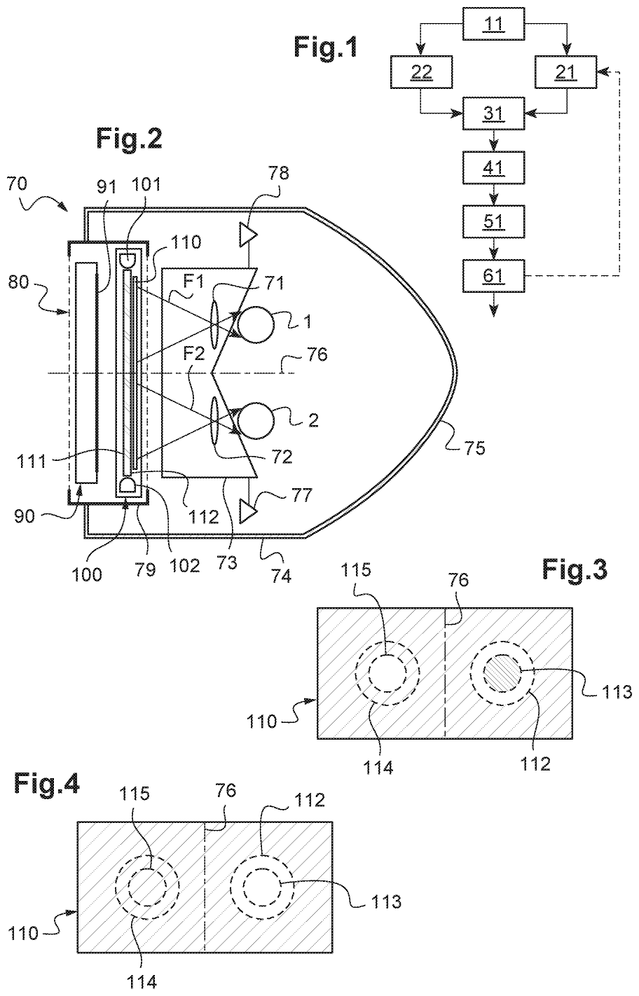

FIG. 2 is a schematic view of a virtual reality headset comprising a smartphone and an additional illuminating system according to a first embodiment;

FIGS. 3 and 4 are detailed views of a scatterer of the additional illuminating system of FIG. 2;

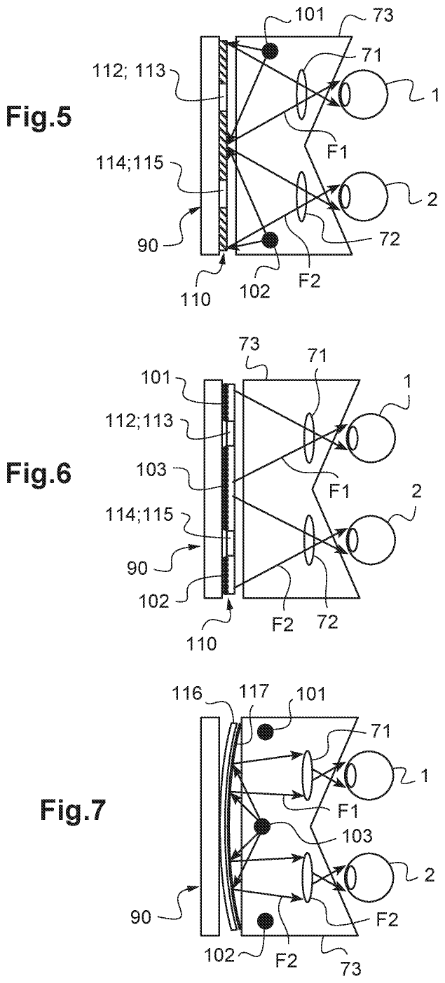

FIG. 5 is a schematic view of an additional illuminating system according to a second embodiment;

FIG. 6 is a schematic view of an additional illuminating system according to a third embodiment; and

FIG. 7 is a schematic view of an additional illuminating system according to a fourth embodiment.

By way of preamble, it will be noted that identical or similar elements of the various embodiments shown in the various figures are referenced with the same reference signs and are not described each time.

Method

FIG. 1 shows a schematic diagram showing the various steps of a determining method according to the invention, which steps will be described in detail below.

This method aims to find a light filter that is optimal for an individual, the function of this filter being to improve or maintain the visual comfort and/or the visual performance of this individual when the latter is wearing a pair of spectacles, to one, and preferably both, of the spectacle lenses of which said optimal filter is applied.

Step a (Block 11 of FIG. 1)

Step a) is a step of collecting data relating to the needs and habits of the individual.

As explained above, the data relating to the “habits” of the individual comprise information on the conditions under which the individual envisions using the light filter and on the possible uses that were mentioned above.

By data relating to the “needs” of the individual, what is in particular meant is any data relating to the visual discomforts or deficiencies of the individual, and optionally to the symptoms that are associated with therewith, and that the sought-after light filter is supposed to palliate, such as those already nonlimitingly mentioned above.

The data relating to the needs of the individual may also comprise information on the personal profile of the individual: his age, his sex, the color of his eyes, any current or past visual pathologies (for example cataracts, macular pigment density, intraocular scattering, ARMD, amblyopia, nystagmus, etc.), his medical history (for example: dyslexia, epilepsy, migraines, autism spectrum disorders), his refraction (for example in the form of data on the power of his optical correction), whether he wears spectacles or contact lenses.

The protection that will be provided by the sought-after light filter aims to improve or maintain his visual comfort (for example: absence of glare) or indeed his visual performance (for example his visual acuity).

By maintaining visual performance, what is also meant is the protection of the eye from harmful radiation such as ultraviolet (UV) radiation, photo-toxic blue light, or indeed infrared (IR) radiation.

The data on the habits and/or needs of the individual allow trial filters that will potentially be of interest to the individual to be preselected.

For example, if the individual for whom a light filter is sought regularly complains of problems with glare, the selection of weakly tinted lenses that a priori will certainly not allow a satisfactory solution to be obtained in terms of visual comfort will be avoided.

It will also be seen that the collected data will allow the visual requirements of the individual to be determined and will assist in the parameterization of the luminous scenes.

For example, if the subject reports an increased incidence of discomfort when driving at night specifically, luminous scenes will be chosen with:
a luminous environment of mesopic or even scotopic type;
point light sources (e.g.: automobile headlights, streetlights, etc.); and/or
a road scene with pedestrians or specific obstacles, with object-recognition or contrast and/or reaction-time tests.

A measurement of the attentional field of the individual will possibly also be taken in order to evaluate accident risk and to evaluate the benefit of the filter on these qualitative and quantitative measurements.

In one particular embodiment, the data are collected by means of a questionnaire.

The individual is then, in step a), asked to respond to this questionnaire, the collected data comprising the responses to the various questions of the questionnaire.

The questionnaire may be carried out by means of a paper medium or indeed a digital medium (for example: computer, tablet, or smartphone). Alternatively, the questionnaire may be carried out in oral form with a practitioner who asks the questions orally and notes the responses, either on a paper medium, or on a digital medium.

The responses to certain questions may be binary (yes/no), or indeed of the type “never/sometimes/often/always”. Sometimes, the responses may be a score, for example one of between 1 and 5.

In another particular embodiment, in step a), in addition to or instead of the questionnaire, measurements relating to the luminous environment of the individual are collected by means of at least one light sensor, the collected data depending on these measurement values.

Step a) may then comprise a step of measuring the light flux to which the wearer is habitually subjected. It is carried out using a light-flux sensor that is independent or integrated into a pair of spectacles or a connected object of the wearer, for example a smartphone, a tablet computer or a connected watch that collect the characteristics of the ambient light flux at the present time. This sensor (spectrophotometer type) allows the characteristics of the light flux to which the wearer is subjected while he is filling in the questionnaire (in particular intensity, spectrum, variation over time) to be collected.

The idea of a connected measurement is to measure the customary light exposure (intensity, spectrum, variations in intensity) of an individual in his habitual environment and to associate them with a degree of glare sensation for example.

The collected data allow the parameters of the trial filter to be chosen depending on the type and the frequency of the glare sensation experienced by the wearer.

It is thus possible to consider a trial filter with a minimum light transmission to be prescribed depending on the light-intensity level from which the individual is discomforted.

Furthermore, if the individual demonstrates a greater visual discomfort to variations in light level, the selection of the trial filter may be directed toward an active filter with a dynamic visual transmission, contrary to a passive filter.

In a first version, provision may be made for a dedicated application on the smartphone of the individual allowing data to be collected each time the smartphone is used. In an improved first version, the data are collected continuously.

The data recorded by the light sensor (for example the photographic sensor of the smartphone) may be: the ambient brightness, the date, the location (position given by the GPS sensor of the smartphone), metrological data and the glare-related discomfort of the subject (via an open question on the degree of glare sensation with a response on a scale from 1 to 5).

Advantageously, provision may also be made for the photo sensor to record an image or a film of the luminous environment in which the individual is found at the moment of his responses. The data thus collected then allow a level of discomfort to be associated with a brightness.

In a second version, provision may be made to use, in addition to the smartphone of the individual, spectacles equipped with a brightness sensor that the individual wears when he responds to the questionnaire.

The ambient brightness is continuously recorded and location information, and glare-sensation indicators, etc. are recorded at regular intervals via the smartphone.

Together, all of the data related to the visual needs and habits of the individual will determine the luminous scenes to which the individual is exposed in step c) of the method (block 31 of FIG. 1).

It will also define the visual requirements of the wearer.

In one preferred embodiment, these elements will possibly then determine the filter-evaluation criteria (step b1 below) or even give indications as to the parameters of the filter to be selected.

Step b1 (block 21 of FIG. 1)

This selecting step b1) consists in selecting at least one trial filter based on at least one predefined evaluation criterion.

In the embodiment of the invention described here, the one or more trial filters are selected depending on the data collected in step a (block 11).

As a variant, provision may be made for the trial filter to be selected among a set of predetermined trial filters.

The objective is to define trial-filter-evaluation criteria that are based either on generic tests, or on the needs or type of environment identified by the individual.

The evaluation criteria may be objective, subjective or mixed.

For example, visual comfort, color perception, or ocular fatigue may be scored on a calibrated scale ranging from 1 (low comfort/poor color perception/high fatigue) to 5 (excellent comfort/very good color perception/low fatigue).

The one or more evaluation criteria may be the result of a measurement of visual performance applied to psycho-physical tests:
visual acuity, sensitivity to contrast, perception of movement or of depth;
reading speed/performance;
recognition of objects in visual scenes, measurement of reaction time in a driving situation;
measurement of the threshold beyond which the individual is discomforted by/sensitive to light.

Lastly, the evaluation criteria may be objective and relate to physiological data linked with visual performance and/or visual comfort such as, for example: dynamic range of the pupil, movement of the eyelids, electro-physiological signal generated by the retina and/or the cerebral cortex (measurable via electro-encephalogram (EEG), electro-retinopathy of the eye (ERG), or visual evoked potentials (VEP)).

The trial filter may be defined by at least one of the following parameters:
value of Tᵥ: percentage of visual transmission in the visible domain under photopic conditions such as defined in standard ISO 8980-3. It is defined in the wavelength range extending from 380 nm to 780 nm as the average weighted by the standard sensitivity V(λ) under photopic conditions of the eye under illuminant D65 (daylight). The visual transmission Tv under scotopic conditions may be defined in the same way with the standard sensitivity V′(λ) under scotopic conditions;
spectral response: for example light-transmission datum as a function of wavelength, in the visible domain between 380 nm and 780 nm, and/or in the ultraviolet domain and/or in the infrared domain;
spatial variation (for example gradient) of the reflectance or absorbance of a lens in the presence of the trial filter (“gradient” filter);
temporal variation in one of the above parameters (for example photo-chromic or electro-chromic filter); or
variation in the polarization state of the transmitted light.

The parameters of the trial filter will possibly be considered one after the other and tested in turn according to the evaluation criteria.

They are pre-selected depending on the data collected in step a (block 11 of FIG. 1).

For example, if the threshold of sensitivity to light of the individual is high, no trial filters of class 0 or 1 as defined in standard NF EN ISO 12312-1 will be tested, but filters with a more protective visual transmission (Tᵥ) will be started with.

Thus, depending on the luminous environments of the individual, certain spectra for which the individual experiences the most discomfort will possibly be preselected.

The parameters of the various trial filters to be selected may also be selected using a preestablished procedure, for example by firstly choosing the visual transmission Tᵥ, then by refining the selection based on spectral responses, and lastly by optionally recommending a photo-chromic or electro-chromic adaptation of the one or more trial filters (if for example a dynamic sensitivity to light is noted).

Step b2 (Block 22 of FIG. 1)

This selecting step b2) (which may take place before or after the selecting step b1) consists in selecting, depending on the data collected in step a (block 11) at least one luminous scene that is representative of the visual habits and/or visual needs of the tested individual.

This luminous scene will serve for the situation exposure of step c (block 31 of FIG. 1).

Advantageously, this luminous scene comprises, on the one hand, an image, or a film, determined depending on these needs and habits, and, on the other hand, at least one light source that generates a discomfort and/or a loss of visual performance for the individual if he does not wear a filter.

Preferably, the image (or the film) of the visual scene is simultaneously defined by at least one of the two following parameters:
an optical or physical parameter of the luminous conditions: intensity (cd), luminance (cd/m²), illuminance (lux), spectrum as a function of the wavelength, temporal variation in these parameters, location and orientation of the source (point or diffuse source), etc.;
a parameter relating to the objects or to the visual activities to be implemented (for example: situation in which a motor vehicle is being driven, walking, reading on a veranda, perception of signs; simple visual stimulus such as a visual acuity letter, a colored or moving target, central vs peripheral stimulus, etc.; no visual stimulus).

All of these parameters are thus able to represent the richness and complexity of the luminous situations with which the individual runs the risk of being confronted.

In other words, the image (or the film) of the luminous scene is a set of objects that are representative of a situation to which the individual could be exposed in real life, said situation being characterized by objects, which are defined by their positions, their sizes and their shapes. For each object, the luminous environment is defined (intensity, spectrum, spatial distribution and temporal variation).

These elements of the environment will allow the individual to be tested in situations close to his needs and also the evaluation criteria of the trial filters to be directed.

In certain embodiments, the image or the film comprises standard visual stimuli, such as those used in conventional optometric tests: visual acuity letter from 1/20 to 20/10 with a contrast between 5% and 100%, test patterns of contrast ranging from 1% to 100% with various spatial frequencies, patterns for testing color vision or depth vision, film for testing the perception of movements or indeed film for simulating a situation in which the individual is walking or driving a motor vehicle, attentional visual field, patch detection.

In order to validate the trial filter with respect to its ability to improve comfort and/or visual performance, the selected luminous scene may also comprise, and, preferably, comprises one or more light sources, each light source generating a discomfort and/or a loss of visual performance for the individual when he is not wearing a filter.

The one or more light sources of the scene are therefore superposed on or added to the image or to the film described above.

Advantageously, provision may be made for it to be possible to select a very high number of light sources depending on the needs and habits noted in step a):
point or point-like source, extended source;
diffuse or directional source;
more or less intense source (level of luminance or of light flux);
colored or uncolored (i.e. substantially white) source, source at T° of warm or cold color;
continuous or intermittent/transitory source, with temporal variation in its parameters (intensity, emission direction, spectrum, etc.);
primary or secondary source;
natural or artificial source; or
source generated by an incandescent, halogen, discharge or vapor (sodium for example) lamp, a light-emitting diode (LED) or an organic light-emitting diode (OLED), etc.

Generally, the additional light source in the image emits an additional light “flux” for the individual observing the luminous scene.

Preferably, provision will be made for the light sources to be able to generate an additional light flux so that the visual luminance of the ensemble formed by the luminous scene and the additional light source has a visual luminance:
higher than 60 cd/m², and preferably higher than or equal to one of the following values: 300 cd/m², 500 cd/m², 1000 cd/m², and 1800 cd/m², when the image of the luminous scene observed only by the individual places him under photopic-vision conditions (average luminance of the image or of the film higher than 10 cd/m²); and
higher than 1 cd/m², under mesopic-vision conditions (average luminance of the image or of the film comprised between 10⁻³ and 10 cd/m²); and
higher than 10⁻³ cd/m², under scotopic-vision conditions (average luminance of the image or of the film lower than 10⁻³ cd/m²).

Under photopic conditions, provision may also be made to vary the additional light flux of the light source so that the illuminance in the plane of the eye is, in one embodiment, at least 200 lux (lx), and preferably higher than or equal to one of the following values: 500 lx, 1000 lx, 1500 lx, 2000 lx, 5000 lx, 10000 lx, and better still 15000 lx. These illuminance ranges correspond to the illuminance values received by the eye in a natural daytime environment when the weather is cloudy to bright.

Also preferably, provision is made to be able to vary the spectrum, the color, and/or the color temperature of the additional light source, the spectrum being able to be narrow or broad, discrete or continuous, mono- or polychromatic.

Advantageously, the level and/or the spectrum of the additional light flux may be programmed by choosing the various light sources (for example various LEDs), or by making the individual wear spectacles with passive or active (electrochromic) lenses the tint, spectral response, and/or activation speed of which may be controlled.

Step c (Block 31 of FIG. 1)

Once the luminous scene and the trial filter have been selected depending on the needs and habits of the individual that were noted in step a), provision is made for a step of exposing the individual to a situation in which the luminous scene is visually presented thereto through the chosen trial filter.

It will firstly be noted that the trial filter may be real and/or virtual. When the trial filter is real, then in step c), the individual actually wears, i.e. physically wears, this trial filter in order to view the luminous scene that is displayed on a displaying device observed by the individual.

In this way, it is possible for the individual to appreciate the impact that the real trial filter has on the displayed luminous scene, and in particular on his visual comfort and/or his visual performance.

As will be described in more detail below in the rest of the description, the displaying device used in step c) is preferably suitable for displaying all the types of luminous scenes described above, and in particular luminous scenes in which the additional light source generates a glare sensation for the individual.

To do this, provision may advantageously be made to use luminous scenes of high dynamic range and a displaying device comprising a high-dynamic-range (HDR) graphical screen capable of displaying this type of luminous scenes.

A real trial filter may for example take the form of a flat colored filter added to an ophthalmic lens or a trial frame or indeed take the form of a tinted lens.

Another type of real filter may be formed by an active electrochromic or indeed liquid-crystal lens.

The trial filter may therefore also be virtual, i.e. nonphysical. What is meant here is that the trial filter, or more precisely the effect of the trial filter, is reproduced by simulation.

In practice, the simulation is carried out by computing the modifications induced by the virtual trial filter (its optical characteristics) on the visual scene (luminance distribution, spectral content, temporal variation, etc.) before the presentation of the luminous scene to the individual.

In other words, in step c), the luminous scene is simulated and displayed by the displaying device through the selected trial filter such as it would be seen by the individual if he were actually wearing said trial filter.

Thus, when the individual observes the displaying device, he perceives the effect of the filter directly on the luminous scene, which is realistically rendered on the displaying device viewed by the individual.

By virtue of this ability to simulate the effects of the trial filter on the luminous scene selected depending on the needs and habits of the individual, it is possible to test a high number of trial filters, and to easily make the optical characteristics of the trial filter vary.

For example, if the trial filter is a solar filter taking the form of a tinted lens, of slightly green color, the benefit of this trial filter on the visual comfort of the individual, for example his sensitivity to glare, will be tested, during the exposure of the individual to a situation in step c), by computing the way in which the spectral content of the luminous scene is modified by the spectral response of the filter.

Advantageously, provision may also be made, before step c), to calibrate the displaying device in order to characterize the properties of its graphical screen in photometric and visual terms: maximum luminance of the screen (in cd/m²), its gamma per channel, its color temperature (in kelvin) or its chrominance value from the white point, its static or dynamic contrast, its spectral characteristics, etc.

Obviously, it is possible in step c) to expose the individual to the situation with two trial filters: a real filter and a virtual filter. To do this, it is enough for the individual to observe the displaying device through the real trial filter, the displaying device simulating and displaying the luminous scene such as it would be seen if the individual were wearing only the virtual trial filter.

Step d (Block 41 of FIG. 1)

During or after step c) of exposure to a situation, the determining method comprises an evaluating step in which the selected trial filter is “scored” with respect to each of the predefined evaluation criteria.

The evaluation of the benefit of the selected trial filter may then be carried out qualitatively and/or quantitatively according to the chosen predefined criterion.

It may also be done either directly by the individual, who himself scores a predefined criterion (for example: the individual gives a score comprised between 1 and 5 for his appreciation of color with the chosen trial filter), or indirectly by the individual who performs a test, the result of this test being the score given to the predefined criterion (for example: result of a visual acuity test or indeed result of a measurement of a retinal recovery time).

For each predefined criterion, the score given by the individual or the result of the indirect evaluation is recorded.

The result of step d) may also be a weighted value of the various scores obtained by the trial filter under test according to various predefined criteria.

The weighting may be a simple average (each criteria has the same weight) or a more complicated weighting, depending on the importance of the needs expressed by the individual.

Step e (Block 51 of FIG. 1)

Once the one or more results of evaluating step d) have been obtained, each is compared to a predetermined visual-comfort and/or visual-performance threshold.

This predetermined visual-comfort and/or visual-performance threshold may be an absolute threshold or indeed a relative threshold.

For example, if the predefined evaluation criterion of the trial filter to be tested relates to its ability to restore a satisfactory visual acuity under glare conditions, the tested trial filter may be considered to have a benefit for the individual if the measured visual acuity (i.e. the result of evaluating step d) is higher than or equal to an absolute threshold of 7/10.

Conversely, it may be desired to compare, in this step, the benefit of the trial filter to a situation in which the individual is wearing no trial filter or indeed a situation in which the individual is wearing another filter that may either be a reference trial filter or indeed simply the trial filter tested beforehand. It is thus possible to adopt an incremental approach whereby an improvement in visual comfort or in visual performance is sought relative to another trial filter previously tested on the individual.

Step f (Block 61 of FIG. 1)

The prescription of the light filter is validated or invalidated on the basis of the result of the comparison of step e).

A targeted validation (comparison with an absolute threshold) or indeed a relative validation (comparison with a relative threshold) of the trial filter may be sought.

If the comparison of step e) demonstrates an improvement in the predefined criterion, then, in step f), said trial filter is adopted as being said light filter suitable for the individual.

In contrast, if the comparison of step e) demonstrates a degradation in the predefined criterion, then, in step f), the determining method is restarted from step b1) with another trial filter being selected (see the dashed arrow between block 61 and block 21 in FIG. 1) and then steps c), d), e) and f) are repeated with this new trial filter. The optimization of the filter may therefore be iterative.

However, the iteration may be carried out even if the comparison demonstrates an improvement in the predefined criterion, so as to tend toward an optimal light filter.

It is possible to imagine introducing a certain tolerance into the comparison of step e), so that a trial filter is considered to be validated or invalidated once the improvement or deterioration, respectively, is larger in absolute value than a predetermined tolerance value c, depending in particular on the measurement precision.

This makes it possible to avoid having to carry out too many iterations for one given predefined criterion and to rapidly converge towards an optimal light filter.

At the end of the method, a light filter that is suitable for the needs and habits of the individual, which needs and habits were noted at the start of the method, will therefore have been determined.

### EXAMPLES

Two examples of application of the determining method such as described above are described below.

### Example 1

It is sought to determine a light filter for an individual who has a visual acuity:
of 9/10 when he is equipped with his visual correction equipment (corrective spectacles); and
of 6/10 under glare conditions, for example under strong sunlight.

It is possible by virtue of the method to test a predetermined series of trial filters on the individual, for example by adding these filters to each of the lenses of his spectacles. The various trial filters have different values of visual transmission Tv and different spectral responses (attenuation as a function of wavelength for example).

By virtue of the displaying device, in step c), a luminous condition beyond the sensitivity threshold of the individual (value measured beforehand) is simulated.

Then, for each trial filter, the visual acuity of the individual is measured. The one or more trial filters that allow a visual acuity of at least 9/10 to be restored in a luminous environment are selected.

### Example 2

An individual having a visual pathology is very sensitive to light and his visual performance is very sensitive to the effect of a light filter. The individual may require a light filter even under low-brightness conditions (indoors situation for example) because the light filter then allows other visual functions to be ameliorated and comfort to be improved.

In this particular case, in step b1), trial filters are selected depending on their spectral response in the visible domain (380-780 nm).

Depending on the specific needs of the individual (which depend on his pathology and on his specific sensitivity), trial filters of different spectral responses will be selected and evaluated according to the following predefined criteria:

A) improvement in clearness;

B) increase in brightness;

C) alteration of colors;

D) protection against light.

The following table collates results obtained by virtue of the determining method of the invention.

This table indicates, by tint range, the first preferred choice of trial filter (spectrum) to be tested first, depending on the criteria of a population of individuals.

For example, if the individual first gives preference to criterion A, i.e. “clearness”, a 450 nm spectral filter (filter F11) will be tested first and the variation in the clearness criterion will be evaluated on a static or dynamic image.

If the subject gives preference to protection against light under indoors brightness conditions, filter No. F15 will preferably be tested in order to then compare it to other filters.

| Criterion A | Criterion B | Criterion C | Criterion D |
|---|---|---|---|
| Filter F11 | Filters F11, | Filters F12, | Filter F15 |
| | F12, F13 | F14 | |

| | |
|---|---|
| If 2nd criterion = C: | If 2nd criterion = A: |
| filters F22, F24 | F21, F′21 |
| If 2nd criterion = D: | |
| filters F21, F22 | |

Device

FIG. 2 shows a virtual reality headset 70 intended to be worn by an individual for whom it is sought to determine an optimal light filter in order to restore or maintain his visual comfort and/or his visual performance.

This virtual reality headset 70 is thus particularly suitable for implementing the determining method of the invention, which method was described above.

On the whole, this headset 70 is a conventional virtual reality headset to which one or more light sources allowing objects or sources of high brightness able to generate a sensation of glare for an individual have been added.

In this case, the individual is immersed in a very realistic luminous environment, which allows luminous scenes of everyday life to be simulated, the movements of the luminous scenes or of glare-generating sources to be realistically simulated and therefore the individual to be able to give rapid and reliable feedback on his level of visual discomfort or visual performance.

As FIG. 2 clearly shows, the virtual reality headset 70 firstly comprises a displaying device 80 intended to test the visual comfort and/or the visual performance of the individual and comprising, to this end, a graphical screen 91 and an additional illuminating system 100.

The graphical screen 91 is, in the embodiment shown here, the display screen of a smartphone 90. It is here controlled by the microprocessor (not shown) of the smartphone 90 in order to display, on this graphical screen 91, an image (or a film) capable of being viewed by the tested individual, who wears the headset 70.

It will be understood here that the image (or the film) is a generated image (or film) of the luminous scene selected in the selecting step of the determining method of the invention.

As a variant, the graphical screen may be a dedicated screen of the virtual reality headset, i.e. one provided with the latter. In this case, the graphical screen may be controlled by a specific microprocessor of the virtual reality headset.

In one preferred embodiment, the microprocessor controls the graphical screen 91 in such a way that this graphical screen 91 displays an image (or a film) formed from a left image (film) for the left eye 2 and a right image (film) for the right eye 1 of the individual.

In this embodiment, the headset 70 also comprises optical means for generating three-dimensional imagery, which means are suitable for presenting the left image and the right image to the left eye of the individual and to the right eye of the individual, respectively, so that the latter sees a three-dimensional image (or film) by fusion of the left and right images (films).

The optical means for generating three-dimensional imagery here comprise (see FIGS. 2, 5, 6, and 7) two lenses, i.e. a right lens 71 and a left lens 72, which are placed in front of the right eye 1 and the left eye 2 of the individual, respectively, and positioned in such a way that the individual sees the right image and the left image displayed on the graphical screen 91 of the smartphone 90.

Advantageously, provision may be made for the two lenses 71, 72 to form intermediate images from the left and right images, these intermediate images being formed in a well-defined plane, for example in a a plane located at optical infinity.

This plane may also be at a near-vision, intermediate-vision or far-vision distance, or at any other distance, depending on the ametropia of the wearer and/or depending on the desire to present a scene at a particular distance for the test.

The additional luminous system 100 is for its part suitable for transmitting, to the individual observing the graphical screen 91, an additional light flux (see arrows F1, F2 in FIG. 2). The individual therefore receives the light flux coming from the images displayed on the graphical screen 91 and the additional light flux F1, F2 emitted by the additional illuminating system 100.

To this end, the headset 70 comprises means 74, 75, 79 for keeping the displaying device 80 in front of the eyes 1, 2 of the individual. In particular, provision is made, on the one hand, for fastenings 79 allowing the displaying device 80 to be attached to the casing 74 of the headset 70 and, on the other hand, for a fastener 75 allowing the individual to fit the headset 70 on his head, the fastener 75 squeezing his head.

The displaying device 80 is fastened to the headset 70 by virtue of the fastenings 79 so that the graphical screen 91 of the smartphone 90 is turned toward the eyes 1, 2 of the individual and so that the additional illuminating device 100 is arranged, with respect to this graphical screen 91, so as to be placed between the latter and the eyes 1, 2 of the individual.

It will be seen below in the rest of the description that the additional luminous system 100 is designed to allow all or some of the image or of the film displayed on the graphical screen 91 of the smartphone to be seen.

The virtual reality headset 70 also comprises a skirt 73 suitable for being pressed against the face of the individual when the latter is wearing the headset 70 with the elastic head strap 75 tightly fastened around the back of his head.

This skirt 73 is opaque and thus allows the individual to be isolated from the ambient light of the environment around the individual.

In this way, during the implementation of the determining method, the individual is placed under controlled luminous conditions, which depend only on the light emitted by the graphical screen 91 (left and right images) and the additional illuminating device 100 (additional light flux F1, F2). The additional illuminating system 100 is controlled by another microprocessor or, as here, by the microprocessor of the smartphone 90. Provision is then made for an interfacing element allowing this microprocessor and the additional illuminating system 100 to interface, so as to allow the latter to receive and process instructions from the microprocessor.

The microprocessor of the smartphone 90 controls the additional illuminating device 100 so that it emits an additional light flux F1, F2 having an energy, a spectral distribution, a temporal variation, a spatial distribution, and/or an angular distribution that is/are predetermined. Various embodiments of the additional illuminating system 100 will be seen in the rest of the description.

Preferably, the additional illuminating system 100 emits an additional light flux F1, F2 allowing a visual luminance higher than or equal to 1000 candelas per square meter (cd/m²), preferably higher than or equal to 2000 cd/m², better still higher than or equal to 3000 candelas per square meter (cd/m²) and preferably of as high as 20000 cd/m² to be achieved.

Such a visual luminance level makes it possible to generate luminous environments in which the additional light flux F1, F2 is liable to generate a visual discomfort for the individual, in particular one due to glare.

Advantageously, the displaying device 80 comprises two light sensors 77, 78 (see FIG. 2) that are suitable for delivering a signal representative of the illuminance level (lux) at the two eyes 1, 2 of the individual, and wherein the average luminance level of the image (or of the film) and/or of the additional light flux F1, F2 is set depending on this representative signal.

Provision may also be made for means 109 for fastening, for example by clip-fastening, the additional illuminating system 100 to the body of the smartphone 90.

As explained above, the additional illuminating system 100 is arranged with respect to the graphical screen 91 so as to allow all or some of the image that it displays to be seen.

To this end, in the first embodiment shown in FIG. 2, the displaying device 80 comprises an additional illuminating system 100 here comprising two series 101, 102 of white light-emitting diodes (LEDs) that are independently controllable and placed on the edge face of a panel 111 made of a transparent plastic, such as polycarbonate or PMMA for example, playing the role of optical waveguide for the light of the light-emitting diodes 101, 102, which light is coupled to the interior of the panel 111.

The additional luminous system also comprises an active or passive scattering film 110 deposited on all or some of the rear face 112 of the luminous panel 111 turned toward the eyes 1, 2 of the individual and suitable for scattering the light guided by total internal reflection in the luminous panel 111.

Advantageously, this scattering film 110 may be an active polymer-dispersed-liquid-crystal (PDLC) film comprising activatable zones allowing light to be scattered or not.

FIGS. 3 and 4 show an example of an active scattering film that may be used in the additional illuminating system 100 of the invention.

In these two figures, the scatterer 110 is formed from a PDLC scattering film of 120 microns thickness sold by Kyushu Nanotec Optics. This active film has two states: a scattering state in the OFF state and a transparent state in the ON state.

The scattering film 110 comprises, for each right and left eye 1, 2 and located on each side of a central axis 76 of the headset 70, two activatable zones 112, 113 and 114, 115:
a central activatable zone 113 (right eye), 115 (left eye); and
a peripheral activatable zone 112 (right eye), 114 (left eye).

These activatable zones 112, 113, 114, 115 are controlled by the microprocessor of the smartphone 90 in order to scatter (“OFF” state) or be transparent (“ON” state) depending on the content displayed on the graphical screen 91.

Advantageously, these activatable zones 112, 113, 114, 115 may, for example, be two apertures allowing stereoscopic vision, with the right and left eyes 1, 2, of the images or film displayed on the graphical screen.

Independent control of these two zones allows vision tests to be carried out independently on the right eye 1 or left eye 2 or on both eyes. The advantage of using a central zone and a peripheral zone is to make it possible to adjust the size of the central zone depending on the size of the luminous scenes to be observed. The zones may therefore be of multiple dimensions.

FIGS. 5, 6 and 7 show schematic views of a displaying device according to a second, third and fourth embodiment, respectively.

In the second embodiment shown in FIG. 5, the additional illuminating system comprises two groups 101, 102 of light-emitting diodes, here of one light-emitting diode, and an active scattering film 110 with the activatable zones 112, 113 and 114, 115 described above.

This scattering film 110 is placed between the eyes 1, 2 of the individual and the graphical screen 91 of the smartphone 90, and backscatters toward the individual the light flux emitted by the light-emitting diodes 101, 102.

In the third embodiment shown in FIG. 6, the additional illuminating system 100 comprises a backlight unit here formed from a matrix array of light-emitting diodes 101, 102, 103, which matrix array is placed in front of said graphical screen, and from an active transmission-mode scatterer 110 that is placed between the backlight unit and the eyes 1, 2 of the individual.

As a variant, the light sources may comprise optical fibers or indeed organic light-emitting diodes (OLEDs).

In the fourth embodiment shown in FIG. 7, the illuminating system comprises three light-emitting diodes 101, 102, 103 and a half-silvered mirror 116 that is placed between the eyes 1, 2 of the individual and the graphical screen 91.

The half-silvered mirror 116 reflects the light flux emitted by the light-emitting diodes 101, 102, 103 toward the eyes 1, 2 of the individual. More precisely, the half-silvered mirror 116 is suitable for projecting onto the cornea or very close thereto, in a plane perpendicular to the normal to the apex of the half-silvered mirror 116, this plane being almost tangent to the apexes of the corneas of the individual.

The advantage of this displaying device, with respect to the preceding ones, is that it makes it possible to benefit from the entire graphical screen 91 while nonetheless being able to act on the light-emitting diodes 101, 102, 103.

In other embodiments, provision could be made for a set of ergonomic controls allowing the individual to make the characteristics of the additional light flux vary by controlling the additional luminous system. Physiological data of the individual (the illuminance levels of the source that create a discomfort for the individual, time to recover clear vision, to recover stereoscopic vision, etc.) could also be recorded.

Provision could be made to prerecord, in the smartphone or the other microprocessor that controls the additional illuminating system and/or the scattering film, a set of protocols for determining light filters. These protocols could make the light intensity and the spectral, spatial and temporal distributions of the one or more additional light sources vary.

Provision could also be made for a “manual” mode, in order to allow the individual to make the light intensity of the displaying device vary. In this case, the displaying device—or headset—may comprise inputting means allowing the individual to express a level of visual discomfort experienced with respect to the additional light flux generated by the additional illuminating system, and means allowing the visual discomfort to be associated with at least one optical characteristic (intensity, spectrum, angular or spatial distribution, temporal variation, etc.) of the additional light flux generated by the additional illuminating system.

In another embodiment, provision may be made to use the displaying device without the virtual reality headset. In this case, the displaying device is then held by the individual at a typical reading distance. A set of light sensors will allow the light level to be adjusted depending on ambient illuminance. The graphical screen may then comprise the screen of a tablet and the additional luminous system may comprise a set of light-emitting diodes placed on the periphery of the screen.

Lastly, in yet other embodiments, the displaying device may be used in an augmented reality headset or in a projecting system.

In the context of an augmented reality headset, the observed luminous scene may be the luminous scene seen by the camera of the smartphone and displayed on the screen thereof, in addition to the glare-generating part of the displaying device.

In the context of a projecting system, powerful projectors could both serve to illuminate and to display the scene to be observed.

The present invention is in no way limited to the embodiment described and shown, but a person skilled in the art will know how to apply thereto any variant in accordance with the spirit thereof.

1. A method for determining a light filter suitable for being applied to a spectacle lens in order to improve or maintain the visual comfort and/or the visual performance of an individual who wears said spectacles, said method comprising:
a) collecting data relating to the needs and/or the habits of said individual;
b1) selecting at least one trial filter based on at least one predefined evaluation criterion;
b2) selecting, depending on said data collected in a), at least one luminous scene, said scene comprising:
an image or a film determined depending on the visual needs and visual habits of the individual; and
at least one light source that generates a discomfort and/or a loss of visual performance for the individual not equipped with filter;
c) exposing said individual to a situation, wherein said luminous scene selected in b1) is visually presented to said individual through said trial filter selected in b1);
d) evaluating said predefined criterion of b1) during the situation exposure of c);
e) comparing the result of the evaluation of d) with a predetermined visual-comfort and/or visual-performance threshold; and
f) determining said suitable light filter depending on the result of the comparison of e).

2. The method as claimed in claim 1, wherein, in b1), said trial filter is selected depending on said data collected in a).

3. The method as claimed in claim 1, wherein, in f), said trial filter is adopted as being said suitable light filter if said comparison of e) demonstrates an improvement in said predefined criterion.

4. The method as claimed in claim 1, wherein, in f), if said comparison of e) demonstrates a degradation in said predefined criterion, the method is restarted from b1) with another trial filter being selected.

5. The method as claimed in claim 1, wherein, in a), said individual is asked to respond to a questionnaire, the collected data comprising responses to said questionnaire.

6. The method as claimed in claim 1, wherein, in a), measurements relating to the luminous environment of the individual are also collected by means of at least one light sensor.

7. The method as claimed in claim 1, wherein, in b1), said trial filter is selected on the basis of at least one of the following predetermined criteria:
visual comfort or sensitivity to glare;
perception of colors or contrasts;
visual acuity;
perception of movements or of depth;
visual field;
reading performance;
visual fatigue;
reaction time;
shape recognition;
pupillary response; or
central and/or peripheral attentional visual field.

8. The method as claimed in claim 1, wherein, in b1), said trial filter is selected depending on one of the following parameters:
a visual-transmission level;
a transmission spectrum in a given wavelength band;
a spatial variation in these parameters over the area of said lens; or
a temporal variation in these parameters over time.

9. The method as claimed in claim 1, wherein, in c):
said luminous scene selected in b2) is displayed by a displaying device;
said individual actually wears said trial filter or else wears said trial filter virtually, said luminous scene then being displayed by said displaying device such as it would be seen by said individual if he were actually wearing said trial filter; and
said individual observes said displaying device.

10. A displaying device intended to test the visual comfort and/or the visual performance of an individual, comprising:
a graphical screen that is controlled by a microprocessor in order to display on said graphical screen an image or a film capable of being viewed by said tested individual,
wherein it also comprises:
an additional illuminating system that is controlled by another microprocessor or by the same microprocessor in order to transmit, to said individual observing said graphical screen, an additional light flux having an energy, a spectral distribution, a temporal variation, a spatial distribution, and/or an angular distribution that is or are predetermined, said additional illuminating system being arranged with respect to said graphical screen so as to let all or some of said image or said film be seen.

11. The displaying device as claimed in claim 10, wherein said additional illuminating system is suitable for transmitting an additional light flux allowing, in combination with said graphical screen, a visual luminance higher than or equal to 1000 candelas per square meter (cd/m²) to be achieved.

12. The displaying device as claimed in claim 10, wherein said additional illuminating system comprises light-emitting diodes and an active or passive scattering film placed between the eyes of said individual and said graphical screen, said scattering film being suitable for backscattering the light flux emitted by said light-emitting diodes.

13. The displaying device as claimed in claim 10, wherein said additional illuminating system comprises a backlight unit placed in front of said graphical screen, and an active or passive transmission-mode scatterer that is placed between said backlight unit and the eyes of said individual.

14. The displaying device as claimed in claim 10, wherein said additional illuminating system comprises at least one light-emitting diode and a half-silvered mirror placed between the eyes of said individual and said graphical screen in order to reflect the light flux emitted by said light-emitting diode toward the eyes of the individual.

15. The displaying device as claimed in claim 10, comprising at least two light sensors that are suitable for delivering a signal representative of the illuminance level at the two eyes of said individual, and wherein the average luminance level of said image or of said film and/or of said additional light flux is set depending on this representative signal.

16. A virtual reality headset intended to be worn by an individual, comprising:
a displaying device as claimed in claim 10;
means for keeping said displaying device in front of the eyes of said individual; and
means for isolating said individual from ambient light.
